(19) United States
(12) Patent Application Publication
(10) Pub. No.: US 2014/0348913 A1
(43) Pub. Date: Nov. 27, 2014

(54) BITTER CRUDE DRUG-COMPRISING GRANULAR PRODUCT

(71) Applicants: KOWA COMPANY, LTD., Nagoya-shi (JP); UMEKEN CO. LTD., Kita-ku (JP)

(72) Inventors: Kazuhiro TANIE, Osaka-shi (JP); Mikio Yokoyama, Chuo-ku (JP); Toru Yuasa, Chuo-ku (JP)

(73) Assignees: KOWA COMPANY, LTD., Nagoya-shi (JP); UMEKEN CO. LTD., Kita-ku (JP)

(21) Appl. No.: 14/282,449

(22) Filed: May 20, 2014

(30) Foreign Application Priority Data
May 27, 2013 (JP) 2013-110665

(51) Int. Cl. A61K 36/9066; A61K 9/20

(52) U.S. Cl. CPC A61K 36/9066; A61K 9/2072; A61K 9/2068
USPC 424/464; 424/756

(57) ABSTRACT

Provided is a granular product that has reduced or suppressed bitterness even though comprising a bitter crude drug or an extract thereof, and that allows a pharmacological action of the bitter crude drug to be sufficiently expressed. The granular product comprises a bitter crude drug or an extract thereof in each of the inner part and outer layer of the granular product.

### FIELD OF THE INVENTION

The present invention relates to a granular product comprising a bitter crude drug or an extract thereof.

### BACKGROUND OF THE INVENTION

In developed countries whose populations have been aging for years, an increasing number of people prefer to utilize Oriental medicine for therapy and prophylaxis of chronic or prolonged diseases. Hitherto, in Oriental medicine, a crude drug has been taken as an infusion or a decoction. However, it is laborious to prepare such dosage form for dosing. Accordingly, in recent years, an extract of a crude drug, which is convenient for dosing, has been widely distributed and frequently used. Among the extracts, solidified ones such as an extract granule and extract powder have been widely used, and as a preparation obtained by using such solidified extract, a tablet or a pill has been used because of its ease of handling.

In Japan, as compared to the tablet, the pill has remained a popular choice because of its ease of handling and ease of dosing and because of its familiarity due to, for example, the fact that the pill has been used since ancient times.

In addition, as the crude drug, there is known zedoary, which is usually obtained by passing a rhizome of Curcuma zedoaria Roscoe (Zingiberaceae) through hot water. Zedoary has a characteristic odor and tastes pungent and bitter as well, and hence is classified as an aromatic bitter stomachic (Non Patent Document 1).

### CITATION LIST

### Non Patent Document

Non Patent Document 1: Commentary of The Japanese Pharmacopoeia, Sixteenth Edition, Hirokawa Shoten Co., D-125-D-127

### SUMMARY OF THE INVENTION

Accordingly, in the case of incorporating a bitter crude drug, such as zedoary, or an extract thereof into a granular product, it is necessary, from the viewpoint of ease of dosing, to design the granular product as a preparation so that the taste and odor derived from the zedoary or extract thereof comprised in the granular product can be masked and the zedoary or the extract thereof can be effectively absorbed in the digestive tract.

An object of the present invention is to provide a granular product that has reduced or suppressed bitterness even though comprising a bitter crude drug or an extract thereof, and that allows a pharmacological action of the bitter crude drug to be sufficiently expressed.

In view of the foregoing, the inventors of the present invention made extensive studies, and as a result, found that bitter crude drug-derived bitterness can be reduced or suppressed and a crude drug component can be allowed to be sufficiently absorbed in the digestive tract when part of a bitter crude drug or extract thereof to be incorporated into a granular product is comprised in the inner part of the granular product and the remaining part of the bitter crude drug or the extract thereof is comprised in the outer layer (including outer surface) of the granular product. Thus, the present invention was completed.

That is, according to the present invention, there is provided a granular product comprising a bitter crude drug or an extract thereof in each of the inner part and outer layer of the granular product.

The granular product according to the present invention has reduced or suppressed bitterness derived from bitter crude drug and allows a crude drug component to be sufficiently absorbed in the digestive tract, thus being excellent in ease of handling and ease of dosing. In addition, the bitter crude drug is present in each of the inner part and outer layer of the granular product, and hence there can be expected immediately effective expression of a pharmacological action and a sustained-release effect.

### DETAILED DESCRIPTION OF THE INVENTION

A granular product of the present invention comprises a bitter crude drug or an extract thereof in each of the inner part and outer layer (preferably outer surface) of the granular product. Hereinafter, the granular product of the present invention and the bitter crude drug or extract thereof to be used in the present invention are described.

The bitter crude drug is not particularly limited. Examples thereof include gambir, aloe, lindera root, rose fruit, corydalis tuber, isodon herb, phellodendron bark, coptis rhizome, zedoary, platycodon root, immature orange, apricot kernel, sophora root, gentian, geranium herb, magnolia bark, evodia fruit, burdock fruit, schisandra fruit, calumba, condurango, saffron, gardenia fruit, cimicifuga rhizome, toad venom, senna leaf, swertia herb, rhubarb, citrus unshiu peel, bitter orange peel, balsam pear, picrasma wood, fritillaria bulb, belladonna herb, sinomenium stem and rhizome, moutan bark, nux vomica, saussurea root, bear bile and Japanese gentian.

The form of such bitter crude drug may be selected as necessary, and the bitter crude drug may be used after being cut or crushed into small pieces or small mass, or being pulverized into powder. For example, zedoary that has been formed into powder is referred to as “zedoary powder”.

In addition, examples of the extract of the bitter crude drug include the so-called “extract” and “tincture”, specifically a liquid obtained by appropriately adjusting the size of the bitter crude drug and adding an extracting agent thereto to perform extraction or a liquid obtained by concentrating the extractive thus obtained on the basis of a known method described in, for example, the General Rules for Preparations of the Japanese Pharmacopoeia, Sixteenth Edition. In addition, there may also be used a product obtained by drying such extract or tincture to make a solid mass, a granule, or powder, e.g., “zedoary dry extract” obtained by drying an extractive of zedoary.

It should be noted that the extract of the bitter crude drug may be prepared on the basis of the above-mentioned known method, but a commercially available product may be used as the extract.

It should be noted that examples of the extracting agent include: a lower (for example, 1 to 7 carbon atoms) monohydric alcohol such as methanol, ethanol or n-butanol; a lower (for example, 1 to 7 carbon atoms) polyhydric alcohol such as ethylene glycol, propylene glycol, 1,3-butylene glycol or glycerin; an ether such as diethyl ether; a ketone such as acetone or ethyl methyl ketone; an ester such as ethyl acetate; a halogenoalkane such as dichloromethane or chloroform; an aromatic hydrocarbon such as benzene or toluene; and water. One kind of those agents may be used alone, or two or more kinds thereof may be used as a mixture. Of those, a lower monohydric alcohol, an ether, a ketone, a mixed liquid of these agents with water or water alone is preferred. Ethanol, acetone, diethyl ether, a mixed liquid of these agents with water or water alone is more preferred. Water-containing ethanol is particularly preferred.

The total content of the bitter crude drug or the extract thereof in the granular product of the present invention may be determined with appropriate consideration of a daily dose depending on, for example, the sex, age and condition of a recipient, and a method of administering the granular product. The daily dose is such an amount that the bitter crude drug or an extract thereof can be taken, in terms of a raw crude drug, in an amount of preferably from 0.05 to 50 g, more preferably from 0.1 to 30 g, even more preferably from 0.3 to 10 g. It should be noted that the content is not limited to the foregoing, and may be appropriately increased or decreased depending on, for example, the sex, age and condition.

In addition, the total content of the bitter crude drug or the extract thereof with respect to the total mass of the granular product of the present invention is preferably from 0.1 to 80 mass %, more preferably from 1 to 60 mass %, even more preferably from 3 to 50 mass %, particularly more preferably from 5 to 40 mass %.

In addition, the granular product of the present invention preferably contains a sweetening agent and/or a taste masking agent in its outer layer from the viewpoints of reducing or suppressing bitterness and the like and improving the ease of dosing. Examples of the sweetening agent include rice syrup and sweet potato. One of the sweetening agents may be used alone, or two or more thereof may be used in combination.

The dosage form of the granular product of the present invention is not particularly limited, but is preferably a granule or a pill from the viewpoint of, for example, the control of the dose of the bitter crude drug.

In addition, the granular product of the present invention, which is in an embodiment containing the bitter crude drug or the extract thereof in both of the inner part and outer layer of the granular product, can be prepared as described below in accordance with a known granulation method or pill manufacturing method described in, for example, the General Rules for Preparations of the Japanese Pharmacopoeia, Sixteenth Edition. First, a granular product is prepared using part of the bitter crude drug or extract thereof to be incorporated into the granular product and as necessary an additive or the like, and then the remaining part of the bitter crude drug or the extract thereof is incorporated as a preparation coating or a pill coating together with, as necessary, the sweetening agent or the taste masking agent.

In addition, examples of the additive include an excipient, a binder and a disintegrating agent.

Examples of the excipient include glycyrrhiza powder, a glycyrrhiza crude extract, starch, dry yeast, a yeast extract, purified lanolin and crystalline cellulose. Examples of the binder include water, glycerin, simple syrup, starch syrup, a glycerin aqueous solution, gum arabic powder, tragacanth powder and sodium carboxymethyl starch.

Because the granular product of the present invention comprises the bitter crude drug or the extract thereof, the granular product can be used as prophylactic and/or therapeutic drugs for various maladies and diseases, foods for specified health use, foods with nutrient function claims and health foods (such as dietary supplements, health supplements, and other supplements) on the basis of a pharmacological action derived from the bitter crude drug.

### EXAMPLES

Hereinafter, the present invention is described in detail by way of Examples. However, the present invention is not limited to Examples shown below.

### Example 1

### Manufacture of Zedoary-Containing Granular Product

Zedoary powder to be incorporated into a granular product was prepared, and part of the total amount of the zedoary powder, balsam pear powder, fish oil and isoflavones (the total amount of which was adjusted so as to be 80 parts by mass in a wet mass) were mixed with a soybean extract, starch, soybean fiber and water. The mixture was kneaded to give a wet mass having appropriate consistency.

The obtained wet mass was formed into a spherical shape to give a granular product. The remaining zedoary powder, rice syrup and sweet potato (Ayamurasaki) powder were added thereto, followed by drying, to give pills each having a diameter of about 7.5 mm. It should be noted that the obtained pills are five pills, containing 80 mg of isoflavones and 569 mg in total of zedoary powder, balsam pear powder and fish oil.

### Test Example 1

### Expression of Pharmacological Effect

In the granular product obtained in Example 1, zedoary is present in both of the inner part and outer surface of the granular product. Therefore, when the granular product is taken, first, zedoary in the outer surface is immediately absorbed, and then the granular product is disintegrated in the stomach and intestines, followed by the release of zedoary from the inner part of the granular product and immediate absorption thereof. Accordingly, there can be expected immediately effective expression of a pharmacological action and a sustained-release effect.

### Test Example 2

### Masking Effect of Granular Product

The granular product obtained in Example 1 does not comprise the total amount of the zedoary in its outer surface, but comprises part of the total amount of the zedoary in the inner part of the granular product. Accordingly, the granular product has less bitterness than a granular product containing the total amount of the zedoary in its outer surface.

In addition, by virtue of the presence of rice syrup and sweet potato powder in the outer surface, the pungent and bitter taste of zedoary is further reduced or suppressed, and hence the ease of dosing is improved.

According to the present invention, it is possible to provide the granular product comprising a bitter crude drug that exhibits a useful pharmacological action, and to reduce or suppress bitterness, which may become a problem in its dosing.

Thus, the ease of handling and ease of dosing are improved, and, by virtue of the presence of the bitter crude drug in each of the outer and inner layers of the granular product, there can be expected immediately effective expression of a pharmacological action and a sustained-release effect.

What is claimed is:

1. A granular product comprising a bitter crude drug or an extract thereof in each of an inner part and outer layer of the granular product.

2. The granular product according to claim 1, further comprising at least one of a sweetening agent or a taste masking agent in the outer layer of the granular product.

3. The granular product according to claim 1, wherein the bitter crude drug is zedoary.

4. The granular product according to claim 2, wherein the bitter crude drug is zedoary.

5. The granular product according to claim 2, wherein the sweetening agent comprises at least one of rice syrup or sweet potato.

6. The granular product according to claim 1, wherein the granular product is in a dosage form of a granule or a pill.

7. The granular product according to claim 2, wherein the granular product is in a dosage form of a granule or a pill.
